(19) United States
(12) Patent Application Publication
(10) Pub. No.: US 2008/0160478 A1
(43) Pub. Date: Jul. 3, 2008

(54) Dental retractor

(76) Inventor: Navid Kia, Skokie, IL (US)

Correspondence Address: Paul E. Schaafsma, NovusIP, LLC, Suite 221, 521 West Superior Street, Chicago, IL, 60610-3135, US

(21) Appl. No.: 11/649,500

(22) Filed: Jan. 3, 2007

(51) Int. Cl. A61B 1/24

(52) U.S. Cl. 433/31

(57) ABSTRACT

In accordance with the principles of the present invention, a dental instrument is provided. A pair of arm members are provided, which is in one embodiment a pair of exam mirrors. Each arm member includes a tissue retaining surface which can be the mirror. The arm members are pivotally connected such that, in use, the tissue retaining surfaces contact tissue in a mouth of a patient.

### FIELD OF THE INVENTION

The invention relates generally to the field of dental instrumentation.

### BACKGROUND OF THE INVENTION

The proximity of oral soft tissue, such as the tongue, cheek and lip, to the field of work can make it difficult to perform dental procedures. In the past, various dental appliances have been used to isolate or open parts of the mouth to facilitate the performance of dental services. Mouth corner spreading devices, also known as cheek retractors or tongur cups, are known in the art for spreading portions of the lips for examination and/or treatment by dental professionals. In addition, tongue retractors, also known as positioners, are known in the art for pressing the tongue out of the way for examination and/or treatment by dental professionals. Such devices facilitate dental and oral surgery procedures by improving visibility and increasing the available working space within a patient's mouth.

Dental props are devices which are inserted into the mouth of a patient between upper and lower teeth to keep the mouth opened in a fixed position while the dental professional is working in the mouth. However, each of these prior art devices suffers from drawbacks.

What would therefore be advantageous would be a dental instrument that retracts oral soft tissues. It would be further advantageous to provide a dental instrument that provides easy access to working area of the mouth for a dental professional while retracting tongue and cheek tissues or tongue and lip. It would be further advantageous to provide a dental instrument that gives a dental professional an extra working hand while administering to a patient. It would be further advantageous to provide a dental instrument that prevents oral soft tissue from getting into working area and getting injured.

### SUMMARY OF THE INVENTION

A dental retractor in accordance with the principles of the present invention retracts oral soft tissues. A dental retractor in accordance with the principles of the present invention provides easy access to working area of the mouth for a dental professional while retracting tongue and cheek tissues or tongue and lip. A dental retractor in accordance with the principles of the present invention gives a dental professional an extra working hand while administering to a patient. A dental retractor in accordance with the principles of the present invention prevents oral soft tissue from getting into working area and getting injured.

In accordance with the principles of the present invention, a dental instrument is provided. A pair of arm members are provided, which is in one embodiment a pair of exam mirrors. Each arm member includes a tissue retaining surface which can be the mirror. The arm members are pivotally connected such that, in use, the tissue retaining surfaces contact tissue in a mouth of a patient.

### BRIEF DESCRIPTION OF THE DRAWINGS

The foregoing aspects and many of the advantages of the present invention will be more readily appreciated as the same becomes better understood by reference to the following detailed description, when taken in conjunction with the accompanying drawings, wherein:

FIG. 1 is a raised, perspective view of a dental retractor in accordance with the principles of the present invention.

FIG. 2 is a raised, perspective view of the dental retractor of FIG. 1 in a different position.

FIG. 3 shows a perspective view of the dental retractor of FIG. 1 is use in a mouth.

### DETAILED DESCRIPTION OF THE INVENTION

While an exemplary embodiment of the invention has been illustrated and described, it will be appreciated that various changes can be made therein without departing from the spirit and scope of the invention.

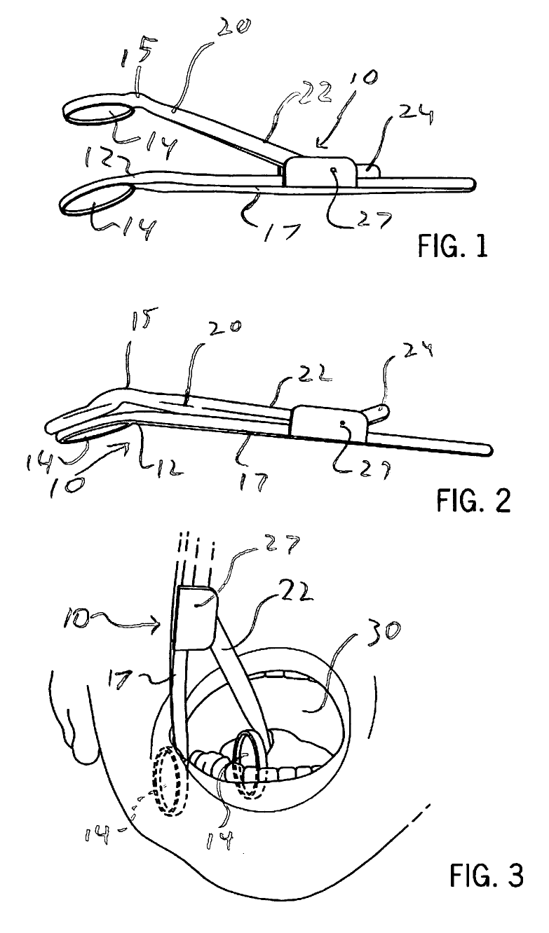

Referring to FIGS. 1-3, a dental retractor 10 in accordance with the principles of the present invention is seen. The dental retractor 10 of the present invention is designed to retract oral soft tissue. The dental retractor 10 of the present invention provides easy access to working area of the mouth for a dental professional while retracting tongue and cheek tissues or tongue and lip. At the same time, the dental retractor 10 of the present invention gives a dental professional an extra working hand while administering to a patient. The dental retractor 10 of the present invention prevents oral soft tissue from getting into working area and getting injured.

Referring to FIG. 1, raised, perspective view of a dental retractor 10 in accordance with the principles of the present invention is seen. The dental retractor 10 of the present invention comprises a pair of arm members 12, 15. In one embodiment, the pair of arm members 12, 15 can comprise a first exam mirror 12 and a second exam mirror 15. The exam mirrors 12, 15 include at one end a mirror 14. In utilizing exam mirrors 12, 15, the mirrors 14 provide a surface that is used to retract oral soft tissue while enabling the dental professional to utilize the mirror for internal mouth vision is desired.

In one embodiment, the first exam mirror 12 includes a handle portion 17 that is generally straight. In one embodiment, the second exam mirror 15 includes a handle portion 20 that is slightly shorter relative to the length of the handle portion 17 of the first exam mirror 12. The handle portion 20 of the second exam mirror 15 includes a first segment 22 and a second segment 24 that is off set relative to the first segment 22 of the handle portion 20 of the second exam mirror 15.

The first exam mirror 12 and the second exam mirror 15 are pivotally connected. In one embodiment, a hinge 27 connects the handle portion 17 of the first exam mirror 12 and the handle portion 20 of the second exam mirror 15. In one embodiment, the hinge 27 is connected to the handle portion 17 of the first exam mirror 12 at about the upper third of the handle portion 17. In one embodiment, the hinge 27 is connected to the handle portion 20 of the second exam mirror 15 at about the junction of the first segment 22 and the second segment 24. In one embodiment, the hinge 27 has a memory component such that the position of the handle portions 17, 20 is generally secure in the absence of manual manipulation. Thus, from a compressed position as depicted in FIG. 2, pressing the handle portions 17, 20 together will separate the mirrors as depicted in FIG. 1. In one embodiment, the hinge 27 can be biased such that the handle portions 17, 20 are generally urged in the separated position, as depicted in FIG. 1. The degree of separation can be adjusted and angles of the mirrors can be modified as well.

Referring to FIG. 3, a perspective view of a dental retractor 10 of the present invention is seen in an example of use in a mouth 30. In use, the dental retractor 10 is put into the mouth 30 in the closed position depicted in FIG. 2 at the position where the dental professional desires to retract oral soft tissue. The dental retractor 10 is put into the open position depicted in FIG. 1 to retract oral soft tissue. As previously described, the mirrors 14 of the exam mirrors 12, 15 provide a surface that is used to retract oral soft tissue while enabling the dental professional to utilize the mirror for internal mouth vision is desired.

While the invention has been described with specific embodiments, other alternatives, modifications and variations will be apparent to those skilled in the art. All such alternatives, modifications and variations are intended to be included within the spirit and scope of the appended claims.

What is claimed is:

1. A dental instrument comprising:
a pair of arm members, each arm member having a tissue retaining surface; and
the arm members pivotally connected such that, in use, the tissue retaining surfaces contact tissue in a mouth of a patient.

2. The dental instrument of claim 1 further wherein the pair of arm members comprises a first exam mirror and a second exam mirror.

3. The dental instrument of claim 1 further wherein the tissue retaining surface comprises a mirror.

4. The dental instrument of claim 1 further including a first handle portion that is generally straight and a second handle portion that is slightly shorter relative to the length of the first, the second handle portion including a first segment and a second segment, one of the segments being off set relative to the other segment, and the first handle portion being pivotally connected at the first segment and the second segment of the second handle portion.

5. A dental instrument comprising a first exam mirror and a second exam mirror, the exam mirrors pivotally connected such that, in use, the exam mirrors contact tissue in a mouth of a patient.

6. The dental instrument of claim 5 further wherein the first exam mirror includes a handle portion that is generally straight and the second exam mirror includes a handle portion that is slightly shorter relative to the length of the handle portion of the first exam mirror, the second handle portion including a first segment and a second segment, one of the segments being off set relative to the other segment, and the handle portion of the first exam mirror being pivotally connected at the first segment and the second segment of the handle portion of the second exam mirror.
